(19) United States
(12) Patent Application Publication
(10) Pub. No.: US 2024/0173013 A1
(43) Pub. Date: May 30, 2024

(54) METHOD AND APPARATUS FOR USING AN INFLATABLE TO ASCERTAIN VESSEL DIAMETER

(71) Applicant: Boston Scientific Scimed, Inc., Maple Grove, MN (US)

(72) Inventors: Ewnet Gebrehiwot, Eden Prairie, MN (US); Paul F. Chouinard, Maple Grove, MN (US); Brady Scott Logan, Monticello, MN (US)

(73) Assignee: Boston Scientific Scimed, Inc., Maple Grove, MN (US)

(21) Appl. No.: 18/521,731

(22) Filed: Nov. 28, 2023

Related U.S. Application Data
(60) Provisional application No. 63/428,640, filed on Nov. 29, 2022.

(51) Int. Cl. A61B 8/08; A61B 6/00; A61B 6/50; A61B 8/12

(52) U.S. Cl. CPC A61B 8/0891; A61B 6/487; A61B 6/504; A61B 8/12

(57) ABSTRACT

An effective blood vessel diameter at a deployment site within a blood vessel may be determined using a measurement apparatus that includes an inflatable balloon. The measurement apparatus may be advanced to the deployment site and the inflatable balloon may be inflated in order to temporarily reshape the blood vessel at the deployment site to a more circular cross-sectional profile. A measurement pertaining to the effective vessel diameter may be obtained while the inflatable balloon remains inflated. The measurement may be obtained using an ultrasound catheter. The measurement may be obtained using fluoroscopy, for example.

### CROSS REFERENCE TO RELATED APPLICATIONS

This application claims the benefit of priority under 35 U.S.C. § 119 of U.S. Provisional Application No. 63/428,640, filed Nov. 29, 2022, the entire disclosure of which is hereby incorporated by reference.

### TECHNICAL FIELD

The disclosure relates generally to medical devices and more particularly to medical devices that are adapted for use in ascertaining vessel diameter for optimal stent selection.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a method of determining an effective vessel diameter at a deployment site within a blood vessel using a measurement apparatus, the measurement apparatus including an inflatable balloon. The method includes advancing the measurement apparatus to the deployment site and inflating the inflatable balloon to temporarily reshape the blood vessel at the deployment site. A measurement is obtained pertaining to the effective vessel diameter while the inflatable balloon remains inflated. The inflatable balloon is deflated and the measurement apparatus is withdrawn.

Alternatively or additionally, the measurement apparatus may further a central lumen adapted to accommodate an ultrasound catheter, and wherein obtaining a measurement pertaining to the effective vessel diameter comprises using the ultrasound catheter to measure the effective vessel diameter.

Alternatively or additionally, inflating the inflatable balloon may cause the central lumen to be centered within the blood vessel.

Alternatively or additionally, the inflatable balloon may further include a radiopaque material sputtered on the inflatable balloon, and the method may further include using fluoroscopy to determine a diameter of the inflatable balloon as another measure pertaining to the effective vessel diameter.

Alternatively or additionally, the inflatable balloon may include a first inflatable balloon and a second inflatable balloon axially spaced from the first inflatable balloon and the central lumen extends between the first inflatable balloon and the second inflatable balloon, and wherein obtaining a measurement pertaining to the effective vessel diameter may include advancing an ultrasound catheter through the central lumen to a point located between the first inflatable balloon and the second inflatable balloon, and using the ultrasound catheter to determine the effective vessel diameter at a point between the first inflatable balloon and the second inflatable balloon.

Alternatively or additionally, the first inflatable balloon and the second inflatable balloon may each include a radiopaque material sputtered thereon, and the method may further include using fluoroscopy to determine a diameter of the first inflatable balloon and/or a diameter of the second inflatable balloon as another measure pertaining to the effective vessel diameter.

Alternatively or additionally, the measurement apparatus may further include a radiopaque surface disposed on the inflatable balloon, and wherein obtaining a measurement pertaining to the effective vessel diameter may include using fluoroscopy to visualize the radiopaque surface and determine a diameter thereof.

Alternatively or additionally, the radiopaque surface may include a sputtered surface.

Alternatively or additionally, the radiopaque surface may include one or more radiopaque rings that encircle the inflatable balloon.

Another example may be found in a measurement apparatus adapted for determining an effective vessel diameter within a blood vessel. The measurement apparatus includes an elongate shaft extending from a proximal region to a distal region, the elongate shaft including a central lumen extending through the elongate shaft and an inflation lumen extending parallel to the central lumen. An inflatable balloon is disposed relative to the distal region of the elongate shaft and fluidly coupled with the inflation lumen, the inflatable balloon adapted to temporarily reshape the blood vessel at the deployment site when the inflatable balloon is inflated. The central lumen is adapted to accommodate an ultrasound catheter extending through the central lumen to a point proximate the inflatable balloon for measuring an effective vessel diameter with the ultrasound catheter disposed within the central lumen.

Alternatively or additionally, the central lumen may be adapted to be centered within the inflatable balloon when the inflatable balloon is inflated.

Alternatively or additionally, the inflatable balloon may be adapted to be visible under fluoroscopy.

Alternatively or additionally, the inflatable balloon may include a radiopaque material disposed thereon.

Alternatively or additionally, the inflatable balloon may include a first inflatable balloon disposed at a first position relative to the elongate shaft and a second inflatable balloon disposed at a second position relative to the elongate shaft that is axially spaced from the first position.

Alternatively or additionally, at least one of the first inflatable balloon and the second inflatable balloon may include a radiopaque material.

Another example may be found in a measurement apparatus adapted for determining an effective vessel diameter within a blood vessel. The measurement apparatus includes an elongate shaft extending from a proximal region to a distal region, the elongate shaft including a central lumen extending through the elongate shaft and an inflation lumen extending parallel to the central lumen. An inflatable balloon is disposed relative to the distal region of the elongate shaft and fluidly coupled with the inflation lumen, the inflatable balloon adapted to temporarily reshape the blood vessel at the deployment site when the inflatable balloon is inflated. The central lumen is adapted to be centered within the inflatable balloon when the inflatable balloon is inflated and to accommodate an ultrasound catheter extending through the central lumen to a point proximate the inflatable balloon for measuring an effective vessel diameter with the ultrasound catheter disposed within the central lumen.

Alternatively or additionally, the inflatable balloon may be adapted to be visible under fluoroscopy.

Alternatively or additionally, the inflatable balloon may include a radiopaque material disposed thereon.

Alternatively or additionally, the inflatable balloon may include a first inflatable balloon disposed at a first position relative to the elongate shaft and a second inflatable balloon disposed at a second position relative to the elongate shaft that is axially spaced from the first position.

Alternatively or additionally, at least one of the first inflatable balloon and the second inflatable balloon may include a radiopaque material.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative measurement apparatus shown disposed within an example blood vessel;

FIG. 2 is a cross-sectional view taken along the line 2-2 of FIG. 1;

FIG. 3 is a schematic view of an illustrative measurement apparatus shown in combination with an ultrasound catheter;

FIG. 4 is a schematic view of an illustrative measurement apparatus shown disposed within an example blood vessel;

FIG. 5 is a schematic view of an illustrative measurement apparatus shown disposed within an example blood vessel;

FIG. 6 is a schematic view of an illustrative measurement apparatus shown disposed within an example blood vessel; and

FIG. 7 is a schematic view of an illustrative measurement apparatus shown disposed within an example blood vessel.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Stents are implanted within the vasculature for a variety of reasons. In some cases, an important consideration when selecting a stent for implantation is properly sizing the stent. If the stent is too small, it may not remain in position. If the stent is too large, this can cause a variety of complications. A potential complication in determining an appropriate size for a stent is that the blood vessels such as veins in which the stent is to be deployed can have an irregular cross-sectional shape. Moreover, because the veins function as a capacitive system, the veins can change in size as a result of changes in blood pressure, for example. These factors can make stent sizing more complicated. In some cases, a vein diameter may be estimated by looking at minimum and maximum cross-sectional dimensions, and taking an average as the diameter.

In some cases, a vein diameter may be estimated by treating the vein as having a circular cross-section. For example, a vein diameter may be estimated by determining the cross-sectional area of the vein, and calculating a diameter from the cross-sectional area using the well-known relationship A=πr² (A is area, r is radius). In some cases, a vein diameter may be estimated by determining the perimeter of a cross-section and calculating a diameter from the perimeter using the well-known relationship P=2πr (P is perimeter). Each of these methods may provide a different diameter estimate for the same vein. Moreover, each of these methods may not appropriately compensate for phasic or positional changes in diameter, oblong or flattened cross-sections, or errors caused by non-perpendicular measurement planes.

In some cases, an expandable element may be disposed within a vessel to be measured, and the expandable element may be allowed to passively expand until the expandable element contacts the vessel walls and at least partially reshapes the vessel. In some cases, the expandable element may be actively expanded. In either event, the goal is to temporarily reshape the vessel in order to obtain a more accurate vessel diameter, not to excessively stretch or otherwise resize the vessel. An estimated diameter of the vessel may be determined by directly measuring the expanded diameter of the expandable element, by using imaging techniques such as ultrasound, or fluoroscopy in instances in which the expandable element is sufficiently radiopaque. An estimated diameter of the vessel may be determined indirectly, such as by measuring one or more other parameters of the expandable element. In some cases, for example when the expandable element is a braid, there may be known relationships between the diameter of the braid and the length of the braid. This means that for a given change in length, a new diameter can be ascertained. In some cases, changes in braid angle may be used to ascertain how the diameter of the braid has changed as the braid has expanded.

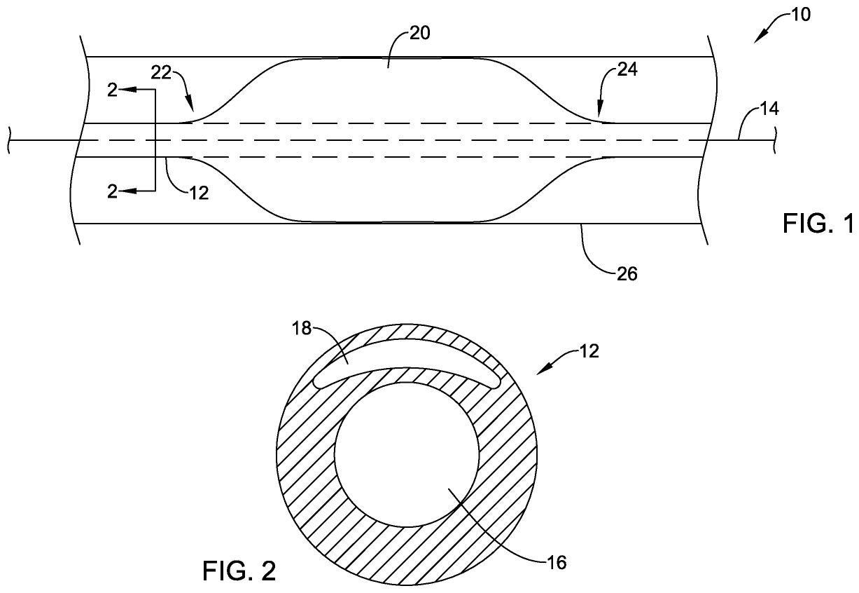

FIG. 1 is a schematic view of an illustrative apparatus 10 that may be used in determining an effective vessel diameter. In this, an effective vessel diameter may be considered as being a diameter that corresponds to the blood vessel having been at least partially reshaped in the process of making the measurement. The reshaping is considered to be temporary, as the vessel will revert to its native profile once the apparatus 10 has been withdrawn. The effective vessel diameter may be considered as being equivalent to a stent diameter of a stent that is an appropriate size to be deployed at that particular location. The stent should have a diameter that is sufficient to at least partially reshape the vessel when implanted, as this will facilitate the stent remaining in position until such time as tissue ingrowth has further secured the stent in place, without overly distending the vessel. A guidewire may be advanced through the vasculature until the guidewire reaches and slightly passes a desired stent deployment site. The apparatus 10 may be advanced over the guidewire until the apparatus reaches the desired stent deployment site so that one or more measurements of a vessel's effective diameter may be taken.

The apparatus 10 includes an elongate shaft 12 that includes a lumen adapted to allow the apparatus 10 to be advanced over a guidewire 14. In some cases, as shown in FIG. 2, which is a cross-sectional view of the elongate shaft 12, the elongate shaft 12 may include a central lumen 16 and an inflation lumen 18. This is merely illustrative, as the internal structure of the elongate shaft 12 may take any of a variety of different configurations. In some cases, the central lumen 16 may be adapted to accommodate the guidewire 14 therethrough. In some cases, the elongate shaft 12 may include a separate guidewire lumen (not shown). In some instances, the central lumen 16 may be considered as being centrally located within the elongate shaft 12 when viewed in cross-section.

Reverting to FIG. 1, the apparatus 10 includes an inflatable balloon 20 that extends from a proximal waist 22 to a distal waist 24. In some cases, at least part of the elongate shaft 12 extends distally through the inflatable balloon 20. As an example, the central lumen 16 may extend distally to at least the distal waist 24. In some cases, the inflation lumen 18 extends at least through the proximal waist 22 such that the inflation lumen 18 may be in fluid communication with an interior of the inflatable balloon 20. This is merely illustrative, as the inflatable balloon 20 may take a variety of configurations.

In some cases, the inflatable balloon 20 may be formed of a polymeric material. In some cases, the central lumen 16 may be disposed within the inflatable balloon 20 such that when the inflatable balloon 20 is inflated, the central lumen 16 is located at or near a center of the inflatable balloon 20. As a result, the central lumen 16 may help to center an ultrasound catheter advanced through the central lumen 16, for example. In some cases, the central lumen 16 may be centered within a cross-section of the elongate shaft 12. In some cases, the central lumen 16 may be off-center with respect to the elongate shaft 12 but may be centered with respect to the inflatable balloon 20, particularly when the inflatable balloon 20 is inflated.

The apparatus 10 is shown disposed within a blood vessel 26. In some cases, the inflatable balloon 20 may be formed of a compliant polymeric material, meaning that when inflated, the inflatable balloon 20 will at least partially deform in response to contacting walls of the blood vessel 26. In some cases, the inflatable balloon 20 may be adapted to at least partially and temporarily deform the blood vessel 26 to make the blood vessel 26 have a more circular cross-sectional profile in the area around where the inflatable balloon 20 is. When the inflatable balloon 20 is subsequently deflated, the blood vessel 26 will revert to its native cross-sectional profile. In some cases, the inflatable balloon 20 may be adapted to provide sufficient force to the walls of the blood vessel 26 in order to cause the blood vessel 26 to be reshaped into a shape that is closer to a round cross-sectional profile while not providing enough force to the walls of the blood vessel 26 to cause the walls of the blood vessel 26 to become stretched.

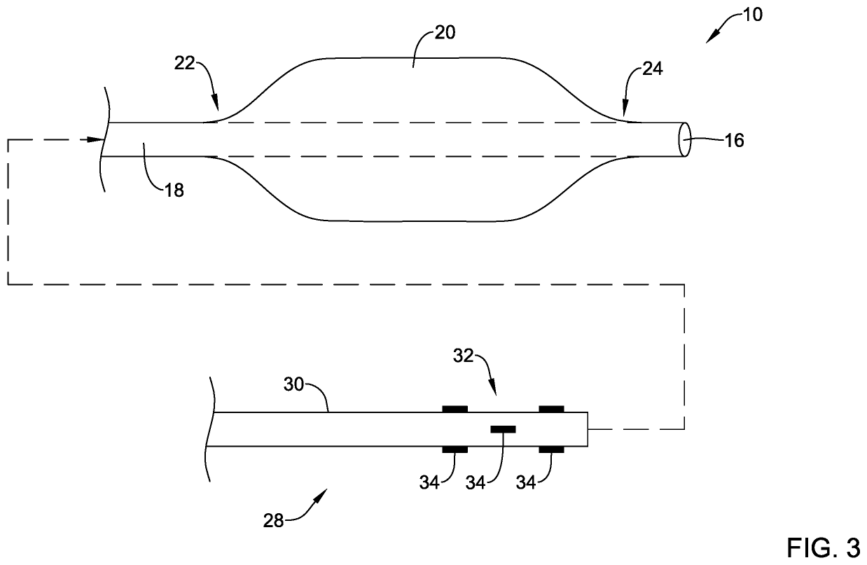

FIG. 3 is a schematic view of the apparatus 10 in combination with an ultrasound catheter 28. The ultrasound catheter 28 includes an elongate shaft 30 extending proximally from a distal region 32. The distal region 32 includes a number of ultrasound transducers 34. The ultrasound catheter 28 may include any number of ultrasound transducers 34. In some cases, the ultrasound transducers 34 may be arranged in pairs about the distal region 32 of the elongate shaft 30, for example. In some cases, the ultrasound transducers 34 may be arranged in other configurations. The ultrasound catheter 28 may be advanced through the elongate shaft 18 and at least partially through the inflatable balloon 20 within the central lumen 16. The ultrasound catheter 28 may be used to ascertain an effective vessel diameter of the blood vessel 26 (FIG. 1) while the inflatable balloon 20 is inflated.

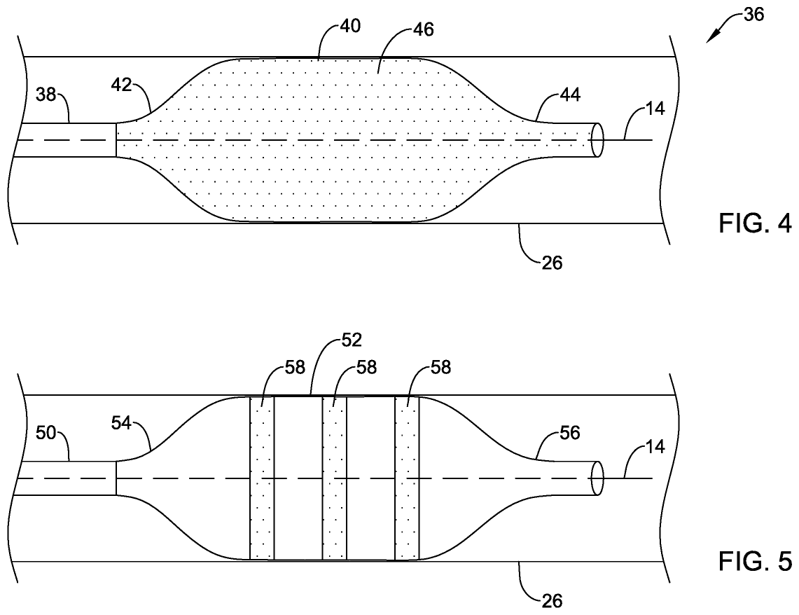

In some cases, fluoroscopy may alternatively or additionally be used in ascertaining an effective vessel diameter of the blood vessel 26. FIG. 4 is a schematic view of an illustrative apparatus 36 shown advanced over the guidewire 14 and disposed within the blood vessel 26. The apparatus 36 includes an elongate shaft 38 and an inflatable balloon 40 that is secured relative to the elongate shaft 38. The inflatable balloon 40 extends from a proximal waist 42 to a distal waist 44. The inflatable balloon 40 may be made of any desired polymeric materials, including polymeric materials that are at least partially compliant.

In some cases, the inflatable balloon 40 includes a radiopaque material 46 that is disposed on an outer surface of the inflatable balloon 40. The radiopaque material 46 is represented as a dot pattern on the inflatable balloon 40. The radiopaque material 46 may be sputtered onto the surface of the inflatable balloon 40. In some cases, the radiopaque material 46 may be coated onto the surface of the inflatable balloon 40. In some cases, the radiopaque material 46 may be integrated into the polymeric material forming the inflatable balloon 40.

In use, the apparatus 36 may be advanced over the guidewire 14 to a position at which an effective vessel diameter measurement is desired for sizing a subsequently implanted stent. The inflatable balloon 40 may be inflated, causing the inflatable balloon 40 to come into contact with the blood vessel 26, at least partially and temporarily reshaping the blood vessel 26 into a more circular cross-sectional profile. Because of the radiopaque material 46 on the inflatable balloon 40, the overall diameter of the inflatable balloon 40 may be ascertained via fluoroscopy. When the inflatable balloon 40 is subsequently deflated, the blood vessel 26 will revert to its native cross-sectional profile. In some cases, the elongate shaft 38 may include a lumen such as the central lumen 16 (FIG. 2) that is adapted to accommodate an ultrasound catheter such as the ultrasound catheter 28. In some cases, fluoroscopy may be used to ascertain the diameter of the inflated inflatable balloon 40 in combination with using an ultrasound transducer to obtain a second measure of the effective diameter of the blood vessel 26.

FIG. 5 is a schematic view of an illustrative apparatus 48 shown advanced over the guidewire 14 and disposed within the blood vessel 26. The apparatus 48 includes an elongate shaft 50 and an inflatable balloon 52 that is secured relative to the elongate shaft 50. The inflatable balloon 52 extends from a proximal waist 54 to a distal waist 56. The inflatable balloon 52 may be made of any desired polymeric materials, including polymeric materials that are at least partially compliant.

In some cases, the inflatable balloon 52 includes one or more bands 58 that include a radiopaque material. While a total of three bands 58 are shown, this is merely illustrative, as the inflatable balloon 52 may include one band 58, two bands 58, or even four or more bands 58. The bands 58 may be sputtered onto the inflatable balloon 52. In some cases, the bands 58 may represent pre-formed metal bands that are subsequently secured in place over the inflatable balloon 52.

In use, the apparatus 48 may be advanced over the guidewire 14 to a position at which an effective vessel diameter measurement is desired for sizing a subsequently implanted stent. The inflatable balloon 52 may be inflated, causing the inflatable balloon 52 to come into contact with the blood vessel 26, at least partially and temporarily reshaping the blood vessel 26 into a more circular cross-sectional profile. Because of the radiopaque material within the bands 58, the overall diameter of the inflatable balloon 52 may be ascertained via fluoroscopy. When the inflatable balloon 52 is subsequently deflated, the blood vessel 26 will revert to its native cross-sectional profile. In some cases, the elongate shaft 50 may include a lumen such as the central lumen 16 (FIG. 2) that is adapted to accommodate an ultrasound catheter such as the ultrasound catheter 28. In some cases, fluoroscopy may be used to ascertain the diameter of the inflated inflatable balloon 52 in combination with using an ultrasound transducer to obtain a second measure of the effective diameter of the blood vessel 26.

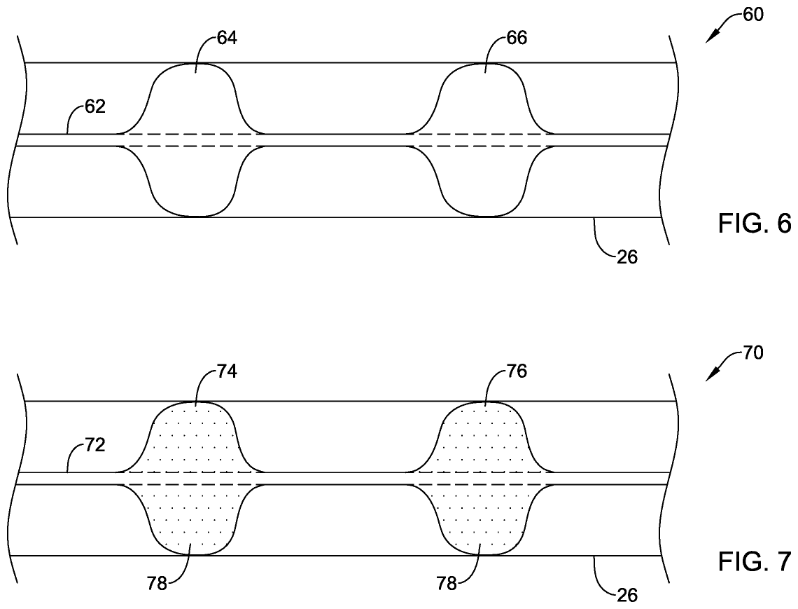

FIG. 6 is a schematic view of an illustrative apparatus 60 shown disposed within the blood vessel 26. The apparatus 60 includes an elongate shaft 62. A first inflatable balloon 64 is secured to the elongate shaft 62 at a first axial position and a second inflatable balloon 66 is secured to the elongate shaft 62 at a second axial position that is spaced from the first axial position. In some cases, the first inflatable balloon 64 and the second inflatable balloon 66 are each formed of a highly compliant polymeric material. The first inflatable balloon 64 and the second inflatable balloon 66 may each be adapted to help temporarily reshape the blood vessel 26 without stretching the walls of the blood vessel 26.

In some cases, the elongate shaft 62 includes an inflation lumen that is in fluid communication with an interior of the first inflatable balloon 64 as well as being in fluid communication with an interior of the second inflatable balloon 66. In some cases, the first inflatable balloon 64 and the second inflatable balloon 66 may each be in fluid communication with their own inflation lumen. This may allow the first inflatable balloon 64 and the second inflatable balloon 66 to be independently inflated and deflated, meaning that an operator may be able to compensate for different profiles or even sizes of the blood vessel 26.

In use, the apparatus 60 may be advanced over the guidewire 14 to a position at which an effective vessel diameter measurement is desired for sizing a subsequently implanted stent. The first inflatable balloon 64 and the second inflatable balloon 66 may be sequentially inflated. The first inflatable balloon 64 and the second inflatable balloon 66 may be simultaneously inflated. Once inflated, each of the first inflatable balloon 64 and the second inflatable balloon 66 will come into contact with the blood vessel 26. In some cases, this may cause a small and temporary amount of reshaping of the blood vessel 26. In some cases, the first inflatable balloon 64 and the second inflatable balloon 66 may have more of a centering action instead of a reshaping action. When the first inflatable balloon 64 and the second inflatable balloon 66 are subsequently deflated, the blood vessel 26 will revert to its native cross-sectional profile

In some cases, the elongate shaft 62 may include a lumen such as the central lumen 16 (FIG. 2) that is adapted to accommodate an ultrasound catheter such as the ultrasound catheter 28. The ultrasound catheter may be advanced through the elongate shaft 62 so that one or more ultrasound measurements may be taken with the ultrasound catheter positioned (or with the ultrasound transducers of the ultrasound catheter) positioned mid-way between the first inflatable balloon 64 and the second inflatable balloon 66.

FIG. 7 is a schematic view of an illustrative apparatus 70 shown disposed within the blood vessel 26. The apparatus 70 includes an elongate shaft 72. A first inflatable balloon 74 is secured to the elongate shaft 72 at a first axial position and a second inflatable balloon 76 is secured to the elongate shaft 72 at a second axial position that is spaced from the first axial position. In some cases, the first inflatable balloon 74 and the second inflatable balloon 76 are each formed of a highly compliant polymeric material. The first inflatable balloon 74 and the second inflatable balloon 76 may each be adapted to help temporarily reshape the blood vessel 26 without stretching the walls of the blood vessel 26.

In some cases, the elongate shaft 72 includes an inflation lumen that is in fluid communication with an interior of the first inflatable balloon 74 as well as being in fluid communication with an interior of the second inflatable balloon 76. In some cases, the first inflatable balloon 74 and the second inflatable balloon 76 may each be in fluid communication with their own inflation lumen. This may allow the first inflatable balloon 74 and the second inflatable balloon 76 to be independently inflated and deflated, meaning that an operator may be able to compensate for different profiles or even sizes of the blood vessel 26.

The first inflatable balloon 74 and/or the second inflatable balloon 76 may include a radiopaque material 78 that is disposed on an outer surface of the first inflatable balloon 74 and/or an outer surface of the second inflatable balloon 76. The radiopaque material 78 is represented as a dot pattern on the first inflatable balloon 74 and the second inflatable balloon 76. The radiopaque material 78 may be sputtered onto the surface of each inflatable balloon 74 and 76. In some cases, the radiopaque material 78 may be coated onto the surface of each inflatable balloon 74 and 76. In some cases, the radiopaque material 78 may be integrated into the polymeric material forming each inflatable balloon 74 and 76.

In use, the apparatus 60 may be advanced over the guidewire 14 to a position at which an effective vessel diameter measurement is desired for sizing a subsequently implanted stent. The first inflatable balloon 64 and the second inflatable balloon 66 may be sequentially inflated. The first inflatable balloon 64 and the second inflatable balloon 66 may be simultaneously inflated. Once inflated, each of the first inflatable balloon 64 and the second inflatable balloon 66 will come into contact with the blood vessel 26. In some cases, this may cause a small and temporary amount of reshaping of the blood vessel 26. In some cases, the first inflatable balloon 64 and the second inflatable balloon 66 may have more of a centering action instead of a reshaping action. Because of the radiopaque material 78, the overall diameter of each of the first inflatable balloon 74 and the second inflatable balloon 76 may be ascertained via fluoroscopy.

When the first inflatable balloon 64 and the second inflatable balloon 66 are subsequently deflated, the blood vessel 26 will revert to its native cross-sectional profile. In some cases, the elongate shaft 72 may include a lumen such as the central lumen 16 (FIG. 2) that is adapted to accommodate an ultrasound catheter such as the ultrasound catheter 28. The ultrasound catheter may be advanced through the elongate shaft 72 so that one or more ultrasound measurements may be taken with the ultrasound catheter positioned (or with the ultrasound transducers of the ultrasound catheter) positioned mid-way between the first inflatable balloon 74 and the second inflatable balloon 76. The measurements ascertained via ultrasound may be used in combination with the diameters captured via fluoroscopy, or one set of measurements may be used as a confirmation of the other set of measurements.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (C) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, The devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method of determining an effective vessel diameter at a deployment site within a blood vessel using a measurement apparatus, the measurement apparatus including an inflatable balloon, the method comprising:
advancing the measurement apparatus to the deployment site;
inflating the inflatable balloon to temporarily reshape the blood vessel at the deployment site;
obtaining a measurement pertaining to the effective vessel diameter while the inflatable balloon remains inflated;
deflating the inflatable balloon; and
withdrawing the measurement apparatus.

2. The method of claim 1, wherein the measurement apparatus further comprises a central lumen adapted to accommodate an ultrasound catheter, and wherein obtaining a measurement pertaining to the effective vessel diameter comprises using the ultrasound catheter to measure the effective vessel diameter.

3. The method of claim 2, wherein inflating the inflatable balloon causes the central lumen to be centered within the blood vessel.

4. The method of claim 2, wherein the inflatable balloon further comprises a radiopaque material sputtered on the inflatable balloon, and the method further comprises using fluoroscopy to determine a diameter of the inflatable balloon as another measure pertaining to the effective vessel diameter.

5. The method of claim 2, wherein the inflatable balloon comprises a first inflatable balloon and a second inflatable balloon axially spaced from the first inflatable balloon and the central lumen extends between the first inflatable balloon and the second inflatable balloon, and wherein obtaining a measurement pertaining to the effective vessel diameter comprises:
advancing an ultrasound catheter through the central lumen to a point located between the first inflatable balloon and the second inflatable balloon; and
using the ultrasound catheter to determine the effective vessel diameter at a point between the first inflatable balloon and the second inflatable balloon.

6. The method of claim 5, wherein the first inflatable balloon and the second inflatable balloon each include a radiopaque material sputtered thereon, and the method further comprises using fluoroscopy to determine a diameter of the first inflatable balloon and/or a diameter of the second inflatable balloon as another measure pertaining to the effective vessel diameter.

7. The method of claim 1, wherein the measurement apparatus further comprises a radiopaque surface disposed on the inflatable balloon, and wherein obtaining a measurement pertaining to the effective vessel diameter comprises using fluoroscopy to visualize the radiopaque surface and determine a diameter thereof.

8. The method of claim 7, wherein the radiopaque surface comprises a sputtered surface.

9. The method of claim 7, wherein the radiopaque surface comprises one or more radiopaque rings that encircle the inflatable balloon.

10. A measurement apparatus adapted for determining an effective vessel diameter within a blood vessel, the measurement apparatus comprising:
an elongate shaft extending from a proximal region to a distal region, the elongate shaft including:
a central lumen extending through the elongate shaft; and
an inflation lumen extending parallel to the central lumen; and
an inflatable balloon disposed relative to the distal region of the elongate shaft and fluidly coupled with the inflation lumen, the inflatable balloon adapted to temporarily reshape the blood vessel at the deployment site when the inflatable balloon is inflated;
wherein the central lumen is adapted to accommodate an ultrasound catheter extending through the central lumen to a point proximate the inflatable balloon for measuring an effective vessel diameter with the ultrasound catheter disposed within the central lumen.

11. The measurement apparatus of claim 10, wherein the central lumen is adapted to be centered within the inflatable balloon when the inflatable balloon is inflated.

12. The measurement apparatus of claim 10, wherein the inflatable balloon is adapted to be visible under fluoroscopy.

13. The measurement apparatus of claim 12, wherein the inflatable balloon comprises a radiopaque material disposed thereon.

14. The measurement apparatus of claim 10, wherein the inflatable balloon comprises:
a first inflatable balloon disposed at a first position relative to the elongate shaft; and
a second inflatable balloon disposed at a second position relative to the elongate shaft that is axially spaced from the first position.

15. The measurement apparatus of claim 14, wherein at least one of the first inflatable balloon and the second inflatable balloon include a radiopaque material.

16. A measurement apparatus adapted for determining an effective vessel diameter within a blood vessel, the measurement apparatus comprising:
an elongate shaft extending from a proximal region to a distal region, the elongate shaft including:
a central lumen extending through the elongate shaft; and
an inflation lumen extending parallel to the central lumen; and
an inflatable balloon disposed relative to the distal region of the elongate shaft and fluidly coupled with the inflation lumen, the inflatable balloon adapted to temporarily reshape the blood vessel at the deployment site when the inflatable balloon is inflated;
wherein the central lumen is adapted to be centered within the inflatable balloon when the inflatable balloon is inflated and to accommodate an ultrasound catheter extending through the central lumen to a point proximate the inflatable balloon for measuring an effective vessel diameter with the ultrasound catheter disposed within the central lumen.

17. The measurement apparatus of claim 16, wherein the inflatable balloon is adapted to be visible under fluoroscopy.

18. The measurement apparatus of claim 16, wherein the inflatable balloon comprises a radiopaque material disposed thereon.

19. The measurement apparatus of claim 16, wherein the inflatable balloon comprises:
a first inflatable balloon disposed at a first position relative to the elongate shaft; and
a second inflatable balloon disposed at a second position relative to the elongate shaft that is axially spaced from the first position.

20. The measurement apparatus of claim 19, wherein at least one of the first inflatable balloon and the second inflatable balloon include a radiopaque material.
